(19) United States
(12) Patent Application Publication
(10) Pub. No.: US 2005/0165367 A1
(43) Pub. Date: Jul. 28, 2005

(54) Vaginal lubrication device

(76) Inventors: Karen D. Higgins, Richmond, VA (US); Kathy Pieper, Midlothian, VA (US)

Correspondence Address: J. MICHAEL MARTINEZ DE ANDINO ESQ.;HUNTON & WILLIAMS, RIVERFRONT PLAZA, EAST TOWER, 951 EAST BYRD ST., RICHMOND, VA, 23219-4074, US

(21) Appl. No.: 10/903,070

(22) Filed: Jul. 30, 2004

Related U.S. Application Data
(60) Provisional application No. 60/539,642, filed on Jan. 28, 2004.

(51) Int. Cl. A61M 31/00

(52) U.S. Cl. 604/279; 604/12

(57) ABSTRACT

The invention provides a device and method for facilitating internal vaginal lubrication. The device is a lubricant delivery device which may be used to deliver lubricant to the vaginal region and accordingly facilitate reduction of sexual friction.

This application claims the benefit of U.S. Provisional Application 60/539,642 filed Jan. 28, 2004, which is incorporated herein by reference.

### BACKGROUND OF THE INVENTION

The invention provides a device to assist women with internal vaginal lubrication.

As the “baby boomer” generation ages, more and more women are moving into the menopausal age range. Lack of vaginal lubrication caused by vaginal atrophy and estrogen deficiency is a major problem within this group of women who want to remain sexually active. While lubricant materials are known and available for external lubrication, there is currently no method or device for conveniently and neatly depositing lubricants in the internal vaginal region. The product Astroglide™ which was marketed after the filing of the provisional application to which this application claims priority provides for internal deposition of lubricants but is in dispensers that have very rigid stems 5 to 8 cm in length. The size and rigidity of the devices make them uncomfortable to use. Further, the known devices dispense from a single orifice at the tip of the stem to a single position. Hence, there remains a need for a convenient comfortable method and/or device for internal vaginal deposition of lubricant materials.

### SUMMARY OF THE INVENTION

The invention provides a device for internal deposition of vaginal lubricant. The device comprises a base portion that includes a base chamber; and a stem portion joined to the base portion. The stem portion includes a central cavity; a stem side; a first stem end with a first end orifice and a second stem end with a second end orifice. One of the first end orifice or the second end orifice joins the base chamber. The device may be constructed of a pliable polymeric material. The device may further comprise at least one side orifice in the stem side and optionally at least one protrusion portion on the stem side wherein the protrusion portion extends from the stem side into the central cavity.

An aliquot of lubricant material may be positioned in the base chamber. Optionally, the exterior of the base portion may be textured. In one embodiment an air tight pressure membrane positioned between the lubricant in the base chamber and the first side orifice of the stem.

The stem portion may be removably or fixedly joined to the base portion.

In an exemplary embodiment the length of the stem portion is less than about 3.5 cm long.

The device of claim 1 wherein the device may be enclosed in sterile packaging.

The invention further includes a method of providing vaginal lubrication comprising:
providing the device described herein
inserting the stem portion of the device into a human vagina; and
squeezing the base portion to deploy the lubricant.

### BRIEF DESCRIPTION OF THE DRAWINGS

FIG. 1 shows a cross-sectional view of one embodiment of the vaginal lubrication device of the invention;

FIG. 2 shows a cross-sectional view of one embodiment of the vaginal lubrication devise of the invention;

FIG. 3 shows a cross-sectional side view of one embodiment of a vaginal lubrication device of the invention;

FIG. 4 shows an external view of one embodiment of the vaginal lubrication device of the invention; and

FIG. 5 shows the distal end of the stem portion of the invention as it would appear when viewing from above the stem portion.

### DETAILED DESCRIPTION OF THE INVENTION

The invention disclosed herein provides a device and method for facilitating internal vaginal lubrication. The device is a lubricant delivery device which may be used to deliver lubricant to the vaginal region and accordingly facilitate reduction of sexual friction caused by vaginal dryness.

Further, the device is designed to minimize messiness and/or discomfort associated with known methods of application of lubricant. Although not required, it is preferable that the device is disposable and packaged individually in sterile packaging. Further, in some embodiments the size of the device is such that it may be packaged in small single units readily amenable to carrying in a handbag or being dispensed from a vending machine, for example.

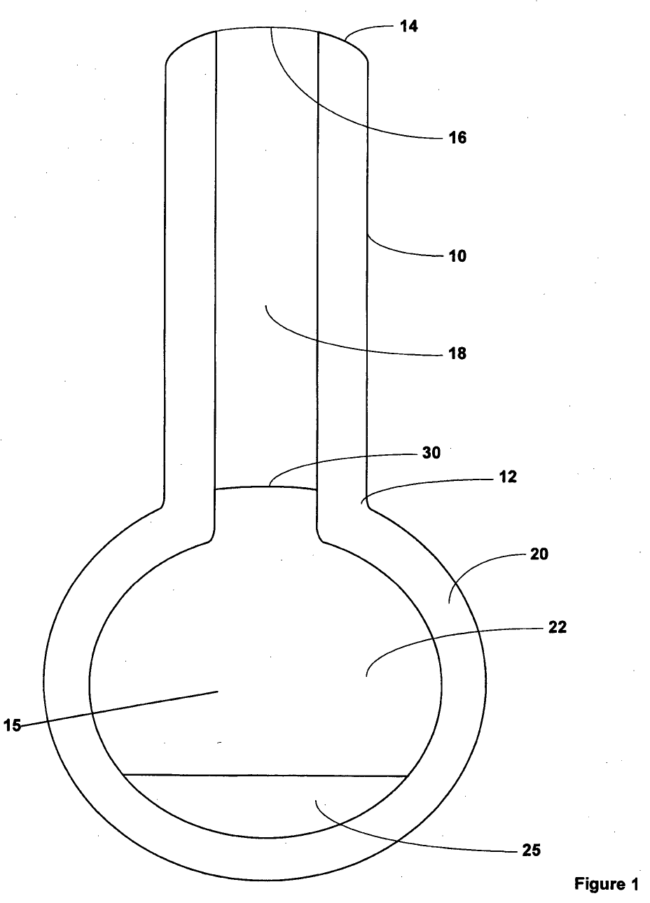

A cross-sectional front view of one embodiment of the vaginal lubrication device is shown in FIG. 1. As FIG. 1 shows, the device includes a stem portion 10 with a central cavity 18 joined to a base portion 20 having a base chamber 22. Lubricant 25 is positioned in the base chamber 22. The volume of lubricant 25 should be sufficient to fill at least some portion of the base chamber 22 and may in some embodiments fill the entire base chamber 22. The base chamber 22 may optionally contain an aliquot of gas 15 to facilitate transfer of the lubricant 25. The gas may comprise, for example, air, an inert gas or some mixture thereof. As one skilled in the art will appreciate, many configurations may be used for positioning lubricant 25 and gas 15 in the base chamber 22, and positioning may depend on the lubricant used and method of preparing the device, for example. In use, the stem portion 10 is inserted into the vagina and the base portion 20 is squeezed to deploy lubricant 25 into central cavity 18 of stem portion 10 and subsequently into the vagina.

The base portion 20 and stem portion 10 may be formed as a single piece or formed separately and fixedly joined together. The size of stem portion 10 should be compatible with comfortable insertion into the vagina and the delivery of the lubricant to the desired position. A variety of sizes are possible. In one exemplary embodiment the stem portion 10 is about 7 mm wide by about 3 to about 3.5 cm long. This embodiment is a size well suited to packaging in single units that may be carried in a handbag or dispensed from a vending machine, for example. The base portion 20 should be sized to facilitate grasping with the hand. In an exemplary embodiment, the base portion 20 is about 2 cm wide and about 2 cm long as viewed from the front as shown in FIG. 1.

It is desirable that the device be constructed of materials that make it compatible with disposal after a single use. Plastic or polymeric materials are examples of suitable materials. It is preferable that the stem portion 10 be constructed of materials with a smooth surface to facilitate insertion. The materials selected for stem portion 10 should be sufficiently rigid to facilitate insertion into the vagina but flexible to minimize discomfort of insertion into the vagina. The material(s) selected for the base portion 20 may be the same or different than the material used for the stem portion 10. The material selected for base portion 20 should have sufficient pliability that it can be squeezed with the human hand during the process of deploying the lubricant.

Further, it is preferable that the exterior surface of the base portion 20 be textured to facilitate grasping. The texturing may be a general texturing pattern or alternatively have decorative aspects.

The lubricant 25 may be any lubricating fluid, cream, ointment, jelly or gel that is compatible with application to sensitive human skin or membranes. Both novel lubricants, as well as known commercially available products, are within the scope of the invention. In one exemplary embodiment, about 2 ml of lubricant is used. The amount of lubricant may be varied and may, for example, be varied depending on the type of lubricant used and particular application. It is preferable that the lubricant 25 is sealed in the base chamber 22 in a sterile condition if the device is prepared substantially remote in time from its use. Sterilization may be accomplished using any suitable method of sterilization known to those skilled in the art.

To preserve the lubricant 25 in a moist state prior to use, a thin air-tight pressure membrane 30 may be placed between the upper surface of the lubricant 25 and below any opening in the stem portion 10. As shown in FIG. 1, the air-tight pressure membrane 30 is positioned approximately at the location where stem portion 10 joins base portion 20. This positioning is exemplary and other positions of the air-tight pressure membrane 30 may be equally suitable.

The air-tight pressure membrane 30 should be constructed of a material that ruptures when the base 20 is squeezed with the force exerted by a human hand. Rupture of the air-tight pressure membrane 30 permits flow of the lubricant 25 into stem 10. Prior to rupture, the pressure membrane 30 should have sufficient integrity to inhibit the passage of air and moisture into and out of the portion of the base chamber 22 containing the lubricant 25.

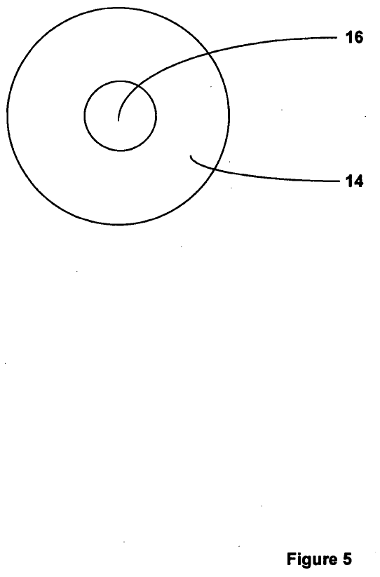

Stem portion 10 has ends 12 and 14 wherein end 12 is adjacent to the base portion 20 and end 14 is distal to base portion 20. The distal end 14 is preferably curved to facilitate the insertion process. The stem portion 10 at distal end 14 has an end orifice 16 adjoining central cavity 18 of the stem portion 10. In use, lubricant is extruded through end orifice 16 into the vagina of the user. In a typical example, the end orifice 16 may have a diameter of about 3 mm. The dimension of the end orifice 16 may be adjusted to optimize deployment of the lubricant in a specific application. FIG. 5 shows the distal end 14 with end orifice 16 as it would appear when viewed from above.

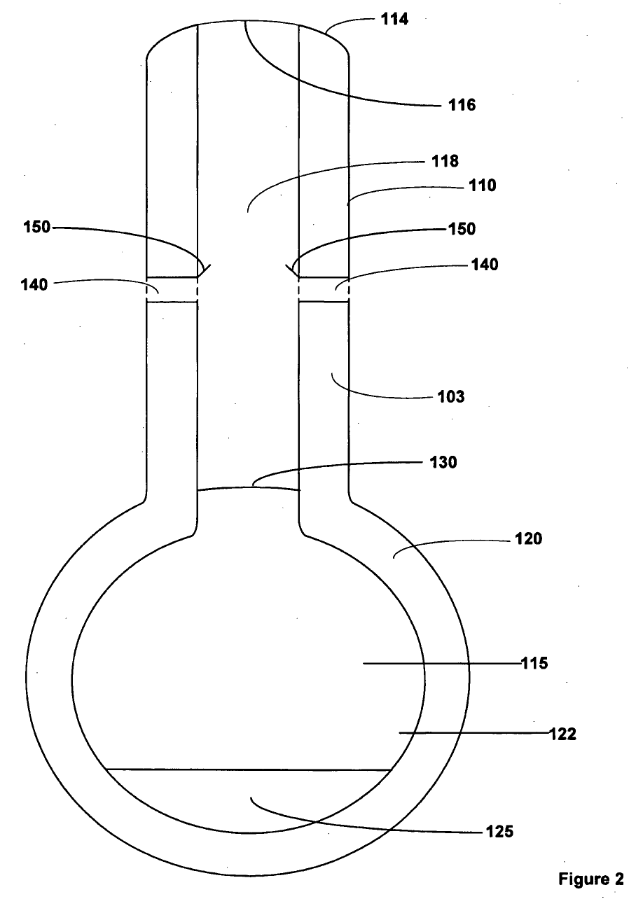

In another embodiment as shown in FIG. 2, the stem portion 110 may have one or more additional side orifices 140 along stem side 103 of stem portion 110. The side orifice permit deposition of lubricant at multiple points in the vagina. The side orifices 140 extend from the central cavity 118 to the external environment surrounding the stem portion 110. These side orifices 140 may be positioned in any of a variety of positions along stem side 103. The exemplary embodiment in FIG. 2 shows two side orifices 140 placed across from each other in a parallel position on stem side 103.

As one skilled in the art will appreciate any number of configurations of numbers and arrangement of side orifices 140 on stem portion 110 may be used in the practice of the invention. However, increasing the number of side orifices 140 along the stem side 103 potentially reduces the total amount of lubricant 125 transmitted and deposited through end orifice 118. Hence, optimal delivery of lubricant 125 should be a guiding factor in determining the position and location of side orifices 140. Likewise, the dimensions of the side orifices 140 may be varied, but it is appropriate to consider the density of the lubricating fluid 125, and amount of lubricating fluid 125 desired to be delivered at a given position in determining the dimensions of the side orifices 140. In one exemplary embodiment the side orifices 140 are about 1 to 2 mm in diameter.

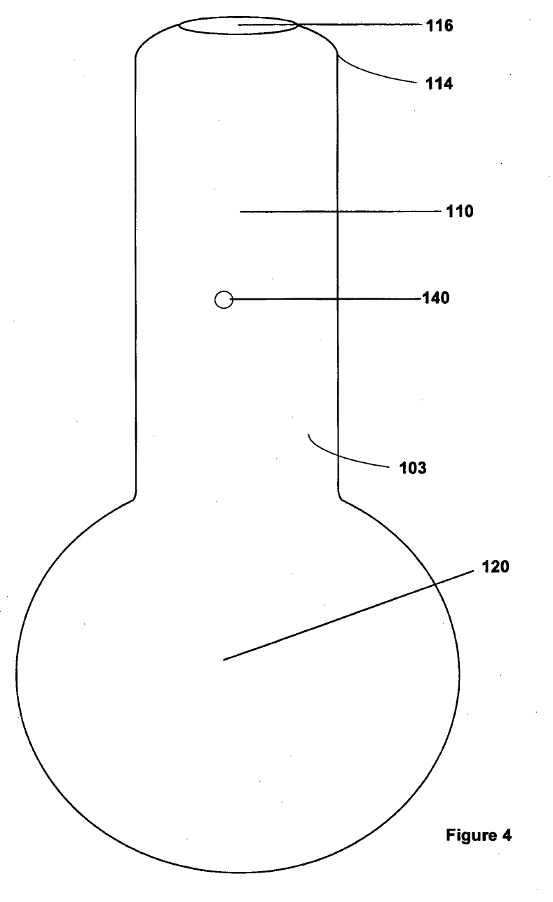

As FIG. 4 shows, the side orifices 140 appear as small openings on the external surface of the stem side 103. FIG. 4 shows an embodiment in which the side orifices 140 are circular. As one skilled in the art will appreciate any number of shapes may be suitable for side orifices 140.

Further, it is preferable that the combination of the size and number of the side orifices and material used to construct the stem 110 are such that sufficient rigidity is maintained for comfortable, convenient insertion. Sufficient rigidity is taken to mean including enough flexibility that the stem will bend if pressed against a surface such as a vaginal wall.

Optionally, as shown in FIG. 2, it may be desirable to have one or more protrusions 150 extending from the stem side 103 into the central cavity 118. The protrusions 150 may be contiguous with the stem side 103 or separate structures fixedly attached. Typically, the protrusions will be positioned at the side of the side orifices 140 nearest the distal end of the stem 114. In the embodiment shown in FIG. 2, each protrusion 150 forms a “v” shape with the stem side 103 with the top of the “v” in the direction of the stem distal end 114. Such structures may facilitate extruding a viscous lubricant through the side orifices 140. As one skilled in the art will appreciate, many designs and configurations could be used for protrusions 150 to facilitate the extrusion of lubricating fluid 125 through orifices 140. The viscosity of the lubricating fluid and the amount that is desired to be deposited at a particular location should be considered in selecting the size and shape of the protrusions 150.

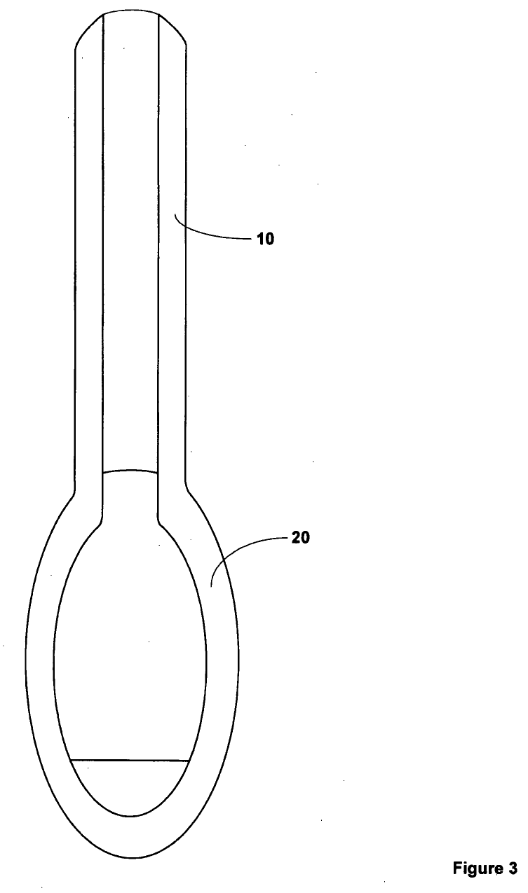

FIG. 3 shows a side view of one embodiment of the device. As FIG. 3 shows optionally the thickness of the base portion 20 may be substantially less than the height and width of the base portion 20 (refer to FIG. 1). Such a configuration may facilitate grasping the base portion 20. The guiding factor should be selection of a size and shape of base portion 20 that is convenient for holding in the human hand, more particularly the size and shape should be convenient for grasping between the thumb and index finger.

In a typical embodiment the device may be presented to the user in packaging. It may be preferable that at least some portion of the packaging holds the device in a sterile condition. Further, it may be desirable that each device be packaged such that one unit (device) may be opened without exposing other units (devices).

To use the device, the user would typically obtain the device in packaging, remove the packaging, insert the stem 10 of the device in the vagina, squeeze the base 20 to dispense the lubricating fluid 25 then remove the device. In one embodiment, the device is disposed of after a single use.

In an alternative embodiment, the stem may be attached to the base by a screw type or interlocking type attachment which may permit the advantage of having one or the other of the stem or base reusable. In some embodiments, a reusable stem may facilitate providing a stem which is particularly well adjusted to a particular user's body conformation and needs. If the stem is to be reused, it would be desirable that it be constructed of materials compatible with effective and convenient cleaning. Further, it may be desirable in some embodiments that a container suitable for storing the stem and holding a cleaning solution in contact with the stem be provided. Providing a reusable base may facilitate delivering an amount of lubricating fluid that is optimum for a particular user. If a reusable base is used, it should be made of a material and shaped in such a fashion that it will be convenient to clean thoroughly. Alternatively, the stem may be adapted to attach to a commercially available lubricant container. If one or the other of the base or stem are reusable such that they are connected for use proximate to the time of use, it is typically desirable to eliminate the pressure sensitive membrane.

Although the present invention has been described in relation to particular embodiments, many variations are possible. Those skilled in the art will recognize that various modifications and variations can be made without departing from the spirit or scope of the invention. The foregoing descriptions are illustrative only and are not intended to limit the scope of the invention in any way.

1. A device for internal deposition of vaginal lubricant, the device comprising:
a base portion, the base portion including a base chamber; and
a stem portion joined to the base portion, the stem portion including a central cavity; a stem side; a first stem end with a first end orifice and a second stem end with a second end orifice;
wherein one of the first end orifice or the second end orifice joins the base chamber; and wherein the device is constructed of a pliable polymeric material.

2. The device of claim 1 further comprising at least one side orifice in the stem side.

3. The device of claim 2 further comprising at least one protrusion portion on the stem side wherein the protrusion portion extends from the stem side into the central cavity.

4. The device of claim 2 further comprising an aliquot of lubricant material positioned in the base chamber.

5. The device of claim 4 further comprising an air tight pressure membrane positioned between the lubricant and the at least one side orifice.

6. The device of claim 1 wherein the stem portion is removably joined to the base portion.

7. The device of claim 1 where in the stem portion is fixedly joined to the base portion.

8. The device of claim 1 wherein an exterior surface of the base portion is textured.

9. The device of claim 1 wherein the length of the stem portion is less than about 3.5 cm long.

10. The device of claim 1 wherein the device is enclosed in sterile packaging.

11. A device for internal deposition of vaginal lubricant, the device comprising:
a base portion wherein the base portion has a base chamber; and
a stem portion joined to the base portion, the stem portion including a central cavity, a stem side, wherein the stem side has at least one side orifice, a first stem end with a first end orifice and a second stem end with a second end orifice wherein one of the first end orifice or the second end orifice joins the base chamber.

12. The device of claim 11 further comprising an aliquot of lubricant material positioned in the base chamber.

13. The device of claim 11 further comprising at least one protrusion portion on the stem side wherein the protrusion portion extends from the stem side into the central cavity.

14. The device of claim 12 further comprising an air tight pressure membrane positioned between the lubricant and the at least one side orifice.

15. The device of claim 11 wherein the stem portion is removably joined to the base portion.

16. The device of claim 11 wherein the device is constructed from a pliable polymeric material.

17. A method of providing vaginal lubrication comprising:
providing the device of claim 1 or 11;
inserting the stem portion into a human vagina; and
squeezing the base portion to deploy the lubricant.
